(19) United States
(12) Patent Application Publication
(10) Pub. No.: US 2004/0101544 A1
(43) Pub. Date: May 27, 2004

(54) Method for detoxifying a carbohydrate containing solution

(76) Inventors: Anders Wieslander, Lund (SE); Torbjorn Linden, Linderod (SE); Reinhold Deppisch, Hechingen (DE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/451,997

(22) PCT Filed: Dec. 28, 2001
(86) PCT No.: PCT/SE01/02915

(30) Foreign Application Priority Data
Dec. 28, 2000 (SE) 0004872-8

(51) Int. Cl. A61K 31/70; C08B 37/00; A61K 47/00

(52) U.S. Cl. 424/439; 514/23; 536/124

(57) ABSTRACT

A device and method for detoxifying a carbohydrate containing solution. A method for identification and determination of the toxicity of a carbohydrate containing medical solution and a carbohydrate containing medical solution as such. A method for detoxifying a carbohydrate containing cell cultivation fluid or food in fluid form; a method for the production of 3,4-dideoxy-glucosone-3-ene and use of a 3,4-dideoxy-glucosone-3-ene detoxifying agent for the production of a medical solution for the treatment of diseases involving disturbed carbohydrate metabolism or compromised removal of carbohydrate degration products.

### BACKGROUND OF THE INVENTION

The present invention relates mainly to a method for detoxifying a carbohydrate containing medical solution, to a device for use in said method, to a method for identification and determination of the toxicity of a carbohydrate containing medical solution and to a carbohydrate containing medical solution as such. The present invention also relates to a method for detoxifying a carbohydrate containing cell cultivation fluid or food in fluid form, to a method for the production of 3,4-dideoxy-glucosone-3-ene, and to use of a 3,4-dideoxy-glucosone-3-ene detoxifying agent for the production of a medical solution for the treatment of diseases involving disturbed carbohydrate metabolism or compromised removal of carbohydrate degradation products.

### BACKGROUND ART

Various types of carbohydrate containing medical solutions are used within medical therapy in the treatment of different diseases and disorders. One example is a dialysis solution being used in the treatment of patients with e.g. renal failure. These patients are generally treated either by extracorporeal haemodialysis (HD) therapies using membrane based dialysers or peritoneal dialysis (PD). During haemodialysis the metabolic waste products are cleared by means of an artificial membrane outside the body, whereas during peritoneal dialysis the waste products are cleared by means of a biological membrane, the peritoneum or peritoneal membrane. In PD the actual exchange takes place between the blood in the peritoneal membrane and the dialysis solution infused. The solution is infused into the abdominal cavity of the patient several times daily through a permanently implanted intraperitoneal catheter. For PD treatment specially designed plastic bags containing a hyperosmotic dialysis solution are used.

A patient on peritoneal dialysis (PD) uses between 8 and 20 litres of dialysis solution every day depending on the treatment. This results in the consumption of 3-7 tons of solution including 1.5-4% of glucose (50-175 kg pure glucose) every year (Wieslander, 1996, Nephrol Dial Transplant 11:958-959), which if the glucose undergoes decomposition also means a non-negligible amount of decomposition compounds. It is well known that some patients experience pain during inflow of the dialysis fluid. It has been speculated that this pain could be the result of glucose degradation products (Henderson et al., 1985 Frontiers in peritoneal dialysis, ed. Winchester, N.Y.: Field, Rich, 261-264) and that the degradation products mediate basal cytotoxicity (Barile F A, 1994, Introduction to in vitro cytotoxicity. Florida: CRC Press, 27-35). This means that they act upon fundamental life processes, which involve structures and functions common to all living cells such as membrane integrity, mitochondrial activity, or synthesis of proteins and DNA. These basal cell functions support organ-specific cell functions. Thus, glucose degradation products (GDPs) capable of affecting basal cell activities are likely to interfere also with specific cell functions such as IL-1 release from mononuclear cells.

Wieslander et al. reported that all major brands of commercial PD solutions were toxic in contrast to PD solutions sterilised by filtration (Wieslander et al., 1991, Kidney Int, 40:77-79). The PD solutions were tested after dilution with cell growth media on cultured fibroblasts. Furthermore, Wieslander et al., have reported that the glucose degradation products also affect the functional responses involved in host defence (Wieslander et al., 1995, Peritoneal Dialysis Int, 15, suppl). Some toxic compounds are known arising from the glucose breakdown, such as formaldehyde, acetaldehyde, metylglyoxal, 3-deoxyglucosone, 5-HMF and glyoxal.

Furthermore, it is well known that patients with diabetes often obtain serious complications namely, diabetic nephropathy or kidney disease, diabetic retinopathy which cause blindness due to destruction of the retina, diabetic neuropathy involving the loss of peripheral nerve functions, and circulatory problems due to capillary damage etc. The similarities between the pathologies arising from diabetes and those resulting from ageing have been extensively reported. A variety of mechanisms have been proposed as a common biochemical basis for both diabetes-associated pathologic conditions and ageing. The hypothesis most strongly supported by data from human subjects is premised on a non-enzymatic glycosylation mechanism. This hypothesis states that the ageing process and diabetes-associated pathologic conditions, such as those described above, are caused, at least in part, by protein modification and cross-linking by glucose and glucose-derived metabolites via the Maillard reaction (Monnier et al., Proc Natl Acad Sci, USA, 1984, 81:583).

It is well known that the Maillard reaction is initiated through the reaction of primary amines (from amino acids, proteins and nucleic acids) with sugars to form Schiff's bases, which undergo rearrangements to form Amadori products. Further rearrangements of the Amadori products are responsible for the browning and fluorescence products, which lead to the formation of advanced glycosylation end products (AGEs). One of the intermediates in the Maillard reaction is 3,4-dideoxy-glucosone-3-ene, for short also called 3,4-DGE. This compound is an intermediate between glucose and 5-HMF (5-hydroxymethylfurfurale). This compound should not be interchanged with the substance 3-DG (3-deoxyglucosone), which is described by Schalwijk C. G. et al in “Induction of 1.2 dicarbonyl compounds, intermediates in the formation of advanced glycation end-products, during heat-sterilization of glucose-based peritoneal dialysis fluids”, Peritoneal Dialysis International, 1999, vol 19, p. 325-333, as an inducer of AGE product formation. 3-DG is a precursor of 3,4-DGE in the glucose degradation. Further, in WO 00/62626 3-DG is pointed out as the toxic substance among the AGE products, and it is suggested to prevent the formation thereof by adding a compound binding to 3-DG or to a precursor thereof.

3,4-dideoxy-glucosone-3-ene has also been reported to have immunosuppressive effects (Kato et al., “Immuno-suppressive effects of 3,4.dideoxyglucosone-3-ene, an intermediate in the Maillard reaction”, J. Agric. Food Chem. 1994, 42, 2068-2073). Furthermore, 3,4-dideoxy-glucosone-3-ene has been reported as a degradation product of fructose (Anet: “Degradation of carbohydrates III”).

So far nobody has managed to identify all of the glucose degradation products in a medical solution, e.g. used for peritoneal dialysis, which are toxic and cause complications or to produce a medical solution of that kind which is non-toxic in this aspect.

Thus, there is a great need for the provision of a method for detoxifying as well as determining the toxicity of a carbohydrate containing medical solution and also the provision of a non-toxic carbohydrate containing medical solution of that kind.

### SUMMARY OF THE INVENTION

The object of the present invention is to eliminate or reduce the above-described problems.

This object is achieved by a method for detoxifying a carbohydrate containing medical solution, wherein a detoxifying agent is contacted with said solution in order to prevent the generation of, reduce the level of or substantially eliminate the amount of 3,4-dideoxy-glucosone-3-ene.

The present invention also relates to a method for identification and determination of 3,4-dideoxy-glucosone-3-ene in a carbohydrate containing medical solution in order to avoid the use of such medical solutions containing the toxic compound 3,4-dideoxy-glucosone-3-ene and thereby reduce complications and undesired side effects caused by 3,4-dideoxy-glucosone-3-ene.

Further, the present invention relates to a device for use in the above-mentioned methods, i.e. for preventing the generation of, reducing the level of or substantially eliminating the amount of 3,4-dideoxy-glucosone-3-ene in body fluids in order to reduce complications caused by 3,4-di-deoxy-glucosone-3-ene.

Moreover, the present invention relates to a non-toxic carbohydrate containing medical solution comprising a detoxifying agent and having reduced levels of or being substantially free of 3,4-dideoxy-glucosone-3-ene.

The present invention also relates to use of a 3,4-dideoxy-glucosone-3-ene detoxifying agent for the production of a medical solution, preferably a dialysis solution, containing carbohydrates for the treatment of diseases involving a disturbed carbohydrate mechanism of compromised removal of carbohydrate degradation products.

In one embodiment the present invention relates to a new method for the production of 3,4-dideoxy-glucosone-3-ene.

The carbohydrate containing medical solution mentioned in connection with the different embodiments of the present invention is necessarily not restricted to a medical solution, although medical solutions are most preferred. Other carbohydrate containing solutions containing an undesired amount of 3,4-dideoxy-glucosone-3-ene may also be applicable. E.g., in another embodiment the present invention refers to a method for detoxifying a carbohydrate containing cell cultivation fluid or a food, preferably in the form of a fluid, wherein a detoxifying agent is added to said fluid or food in order to prevent the generation of, reduce the level of or substantially eliminate the amount of 3,4-dideoxy-glucosone-3-ene.

In still another embodiment the present invention relates to a method for detoxifying the body fluids of a mammal having 3,4-dideoxy-glucosone-3-ene in its body fluids, wherein the body fluids including 3,4-dideoxy-glucosone-3-ene are preferably extracorporeally added to a membrane or column having coupled thereto the detoxifying agent for the binding of 3,4-dideoxy-glucosone-3-ene.

Moreover, in still another embodiment the present invention relates to a method for the treatment of a mammal suffering from a disease condition adversely activating the immune system, wherein a non-toxic amount of 3,4-dideoxy-glucosone-3-ene is administered to the mammal as an immunosuppressive agent.

More specifically, in the method for detoxifying a carbohydrate containing medical solution according to the present invention said solution is contacted either directly with the detoxifying agent in the solution, said detoxifying agent being optionally bound to beads or particles, or with a column or membrane, such as a polymer column or polymer membrane, having coupled thereto a detoxifying agent, i.e. a 3,4-dideoxy-glucosone-3-ene binding agent, for binding 3,4-dideoxy-glucosone-3-ene to the column or membrane and withdrawing the carbohydrate containing medical solution with a reduced amount of 3,4-dideoxy-glucosone-3-ene.

As to the device embodiment for determining the toxicity of a carbohydrate containing medical solution, food, or cell culturing solution, further embodiments of the present invention are;

a membrane comprising the detoxifying agent, i.e. a 3,4-dideoxy-glucosone-3-ene binding agent, for binding 3,4-dideoxy-glucosone-3-ene; and

a column comprising the detoxifying agent, i.e. a 3,4-dideoxy-glucosone-3-ene binding agent, for binding 3,4-dideoxy-glucosone-3-ene.

The carbohydrate in the carbohydrate containing medical solution or cell culturing solution or food fluid is glucose, fructose, mannose, a polymer or derivative thereof, or mixtures thereof, preferably glucose.

The present invention is based on the surprising discovery by the present inventors that 3,4-dideoxy-glucosone-3-ene represents a novel GDP in a medical solution and that it is extremely cytotoxic and is the main candidate to be held responsible for the clinical bioincompatibility in carbohydrate containing medical solutions. It was also found that removal of 3,4-DGE from carbohydrate containing medical solutions strongly reduces the adverse side effects normally associated with administration of such solutions to mammals, even though all other more or less toxic compounds are still present in the solution.

Due to the present invention it is now possible to identify one of the most, if not the most, toxic substances in a carbohydrate containing medical solution in order to remove said toxic substance from the solution or discard the entire solution. It is thereby possible to prevent patients, being treated with such a solution, from experiencing pain or other complications related to said toxic substance, which in turn results in enhanced life quality for the patient.

Furthermore, it is by means of the present invention possible to identify and discard a carbohydrate containing medical solution including said toxic compound before it is used in the treatment of a patient, which makes the overall treatment more efficient and economical.

### BRIEF DESCRIPTION OF THE DRAWINGS

FIG. 1 is a chromatogram of a standard solution with 3,4-dideoxy-glucosone-3-ene obtained with a first method,

FIG. 2 is a chromatogram of a 4% PD solution obtained with a first method,

FIG. 3 is a chromatogram of a standard solution with 3,4-dideoxy-glucosone-3-ene obtained with a second method,

FIG. 4 is a chromatogram of a 4% PD solution obtained with a second method and

FIG. 5 is a dose response curve of 3,4-dideoxy-glucosone-3-ene in a cell growth medium.

### DETAILED DESCRIPTION OF THE INVENTION

As appears from above, the present invention concerns identification of the substance 3,4-dideoxy-glucosone-3-ene in a carbohydrate containing medical solution and particularly the identification of such a compound in a glucose containing medical solution, but also to the determination of the toxicity of 3,4-dideoxy-glucosone-3-ene and to the detoxification of a carbohydrate. containing medical solution.

The inventors have found 3,4-dideoxy-glucose-3-ene as a highly toxic degradation product in a carbohydrate containing medical solution, and in a cytotoxicity test it has been found that the 3,4-dideoxy-glucosone-3-ene is toxic to fibroblast cells. 3,4-dideoxy-glucosone-3-ene was found to be more toxic than other known toxic compounds, such as formaldehyde, acetaldehyde, metylglyoxal, 3-deoxyglucosone and glyoxal, produced during sterilisation and/or storage of a glucose solution. None of these five listed compounds show toxicity in the L-929 system tested, neither alone nor in mixtures at the concentrations found in PD fluids (Wieslander et al, 1995, Peritoneal Dialysis Int., 15:348-352; Witowski et al, 200, J. Am. Soc. Nephrol, 22: 729-739).

Some of the terms and expressions used throughout the specification and the claims are defined in the following.

According to the invention the term “3,4-dideoxy-glucosone-3-ene” is intended to cover both its cis and trans isomers, represented by the following chemical formulas,

3,4-dideoxy-glucosone-3-ene (cis)

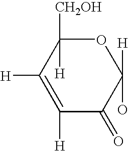

3,4-dideoxy-glucosone-3-ene (trans)

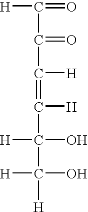

The term “carbohydrate containing medical solution” is intended to mean any medical solution in which 3,4-dideoxy-glucosone-3-ene, its cis and/or trans isomers, is produced as a decomposition compound from the carbohydrate(s) during sterilisation and/or storage of the medical solution, e.g. solutions intended for dialysis. One example of a carbohydrate containing medical solution according to the invention is a glucose containing medical solution present in one or more compartments used for peritoneal dialysis. Preferably the medical solution is a sterile medical solution.

The term “carbohydrate” is intended to mean any carbohydrate producing 3,4-dideoxy-glucosone-3-ene as a degradation product, such as glucose, fructose and mannose , a polymer thereof, natural or synthetic, or derivatives or mixtures thereof.

The term “glucose” is intended to mean a glucose molecule, a polymer of glucose, natural or synthetic, or derivatives or mixtures thereof.

The term “detoxifying agent” is intended to mean an agent with the ability to prevent the generation of 3,4-dideoxy-glucosone-3-ene, to reduce the amount of or substantially eliminate by degrading, scavenging and/or modifying 3,4-dideoxy-glucosone-3-ene.

The detoxifying agent is preferably selected from a group of substances comprising amino, e.g. acetylcysteine, sulphite, and/or sulphydryl groups or pharmaceutically acceptable substances, e.g. organic or inorganic acids, such as HCl and CO₂,giving a very low pH, preferably a pH of around 3. Alternatively, in the case the detoxifying agent is bound to beads or small particles, or coupled to a membrane or column, the detoxifying agent may also be selected from the group comprising polystyrene divinyl benzene polymers and octadecyl silica or a combination thereof may be used. Furthermore, specific and active enzymes for the conversion of 3,4-dideoxy-glucosone-3-ene may be used.

Preferably the detoxifying agent is a sulphite containing compound, e.g. any monosulphite ion (SO₃²⁻), hydrogen sulphite ion (HSO₃—) or disulphite ion (S₂O₃²⁻) bound to a cation, such as sodium, potassium, calcium, magnesium and ammonium. Examples of useful sulphite compounds are NaHSO₃, Na₂S₂O₅ and Na₂SO₃ or any other sulphite compound or derivative thereof, natural or synthetic, or mixtures thereof.

The amount of detoxifying agent to be added is dependent on the amount of carbohydrate in the solution and will easily be defined by the skilled man in the art.

If a monosulphite compound is used, it may be added in an amount to give a final concentration of monosulphite compound in the final solution of 0.001-10 mM, preferably 0.001-0.2 mM, more preferably 0.001-0.1 mM. The same concentrations apply for a hydrogen sulphite compound. If a disulphite compound is used it is preferably added to give a final concentration of disulphite compound within the range of 0.0005-5 mM, preferably 0.0005-0.1 mM, more preferably 0.0005-0.05 m.

The term “detoxifying” is intended to mean preventing the generation of 3,4-dideoxy-glucosone-3-ene, reducing the amount of or substantially eliminating by degrading, scavenging and/or modifying 3,4-dideoxy-glucosone-3-ene produced, e.g. during sterilisation and/or storage of carbohydrate containing medical solutions. The term “substantially. eliminating” means that some trace amounts of 3,4-dideoxy-glucosone-3-ene may remain in the solution after the detoxification. The highest concentration recommendable of 3,4-DGE is approximately 0.7 μM in 4% glucose solution. No biological effects seem to be present at a concentration below the magnitude of 2 μM 3,4-dideoxy-glucosone-3-ene.

The term “compartment container comprising a medical solution” is intended to mean any container comprising one or more compartments, particularly two or three, but not limited to three compartments. One example is a multiple compartment container used for peritoneal dialysis solutions.

The term “peritoneal dialysis solution” is intended to mean a solution comprising an electrolyte, a buffer and an osmotic compound, wherein the electrolyte comprises such ions as sodium, potassium, calcium and magnesium ions; the buffer comprises such components as acetate, lactate or bicarbonate or a mixture thereof; and the osmotic compound is a carbohydrate as defined above. Moreover, any other pharmaceutically aceptable additive may be present in such a solution.

The term “polymer” is intended to mean a polymer as such or in the form of a column, designed to be able to bind reversibly or irreversibly to 3,4-dideoxy-glucosone-3-ene.

The term “membrane” is intended to mean a flat or hollow fibre membrane with the ability to bind reversibly or irreversibly to 3,4-dideoxy-glucosone-3-ene.

The term “sulphite” is intended to mean monosulphite, hydrogen sulphite, and/or “disulphite”.

The term “body fluid” is here intended to mean any body fluid which may contain 3,4-dideoxy-glucosone-3-ene, preferably blood or peritoneal fluid.

Materials and Methods

Identification and Determination of 3,4-dideoxy-glucosone-3-ene

3,4-dideoxy-glucosone-3-ene may be identified and determined by conventional HPLC using a C₁₈ column or HPX-87H column. Two different HPLC methods for determining 3,4-dideoxy-glucosone-3-ene will be described; one using reversed phase (RP) chromatography on a C18 column (method 1) and one using an ion exchange column (method 2), both with diod array detection. According to Anet; Aust. J. Chem. 1962:15;503-509 the cis form of 3,4-dideoxy-glucosone-3-ene has an absorption maximum at 228 nm and the trans form at 233 nm. This was used for identification since these absorption characteristics are typical for alfa-beta ketones. The first mentioned method was also adapted to a mass-detector to confirm the molecular mass.

Other possible methods (not described in the present application) for detecting 3,4-dideoxy-glucosone-3-ene are thin layer chromatography, NMR and UV spectroscopy.

In Vitro Assay for Cytotoxicity.

Carbohydrate containing medical solutions used for peritoneal dialysis were mixed with 1 part of a cell growth medium and 10% (volume/volume) fetal calf serum was added (Wieslander et al., 1991, Kidney Int. 40:77-79). The basal cytotoxicity of the medical solution used for peritoneal dialysis was determined on mouse fibroblasts cells L-929 (CCL-1; ATTC, Rockville, Md., USA) as described earlier (Wieslander et al. 1993, Advances in Peritoneal Dialys, 9:31-35) and expressed as inhibition of cell growth (ICG).

Method for Detoxifying a Carbohydrate Containing Medical Solution

In a preferred embodiment of the method according to the present invention for detoxifying a carbohydrate containing medical solution, wherein a detoxifying agent, e.g. a 3,4-DGE binding agent, is contacted, directly in solution or coupled to a membrane or a column, with the carbohydrate containing medical solution, the contact with the detoxifying agent being performed prior to and/or after a sterilisation process, preferably prior to sterilisation.

The carbohydrate containing medical solution may be sterilised by any kind of sterilisation, such as heat, pressure or radiation, e.g. UV radiation, radioactive radiation, or radiation using micro waves. Preferably the method is used for preparation of carbohydrate containing medical solutions used for peritoneal dialysis, such as a glucose containing solution being heat sterilised.

The carbohydrate containing medical solution may be used in a multiple compartment container for peritoneal dialysis, such as a three-compartment container.

Examples of a carbohydrate containing medical solution for use as a peritoneal dialysis solution could be found in Wieslander et al., 1991, Kidney Int 40:77-79. The peritoneal dialysis solution could further include other pharmaceutically acceptable additives. The peritoneal dialysis solution could prior to dialysis be present in one or more compartments. In the case of multiple compartments the solutions are mixed prior to peritoneal dialysis.

The method may also be used for the preparation of a solution in a multiple compartment container used for peritoneal dialysis, wherein the detoxifying agent may be added either to the glucose containing solution in one compartment or to the electrolyte solution in the other compartment of a multiple compartment. Alternatively, the detoxifying agent may be added to both the glucose containing solution and the electrolyte solution.

The detoxifying agent may be added to the carbohydrate containing solution directly as such or as immobilised on beads or small particles, wherein the detoxifying agent reacts with and/or binds to the 3,4-dideoxy-glucosone-3-ene in the carbohydrate containing solution.

The products obtained in the reaction between the detoxifying agent and the 3,4-dideoxy-glucosone-3-ene when the detoxifying agent is immobilised on beads or particles, may be filtered away by use of conventional filter means before the carbohydrate containing solution, e.g. a peritoneal dialysis solution, is administered to the body.

Alternatively, the detoxifying agent may be bound to membranes or columns arranged between the different compartments in a multiple compartment container or in tubes through which the carbohydrate containing solution, e.g. a peritoneal dialysis solution, is administered to the body.

In a test the PD solution Gambrosol™ trio containing 4% glucose was detoxified by means of an Isolut ENV column.

The tested PD solution contained 0.54 μM 3,4-DGE which corresponds to 0.078 ppm 3,4-DGE (see the table below).

| ml Gambrosol ™ trio | ppm 3,4-DGE |
|---|---|
| 0 | 0.078 |
| 10 | 0.028 |
| 40 | 0.056 |
| 100 | 0.073 |

When detoxifying 10 ml Gambrosol™ trio by means of letting it pass the ENV-column the amount of 3,4-DGE was lowered to 0.028 ppm. The test confirmed that a medical solution, even when already containing a small amount of 3,4-DGE may be further detoxified by means of the invention. The detoxifying effect is reduced when the amount of solution to be detoxified is increased. However, this depends on the limited effect of the column used in this specific example. An increased scale of the column will give the same effect on the increased amounts of solution to be detoxified.

Carbohydrate Containing Medical Solution

The carbohydrate containing medical solution which has been detoxified according to the invention as defined above comprises reduced levels of or is substantially free from 3,4-dideoxy-glucosone-3-ene. In a 4% glucose solution it may be present in an amount of less than 0.7 μM. As described above, 3,4-dideoxy-glucosone-3-ene is produced during the decomposition of carbohydrates, such as glucose and/or fructose, e.g. during sterilisation and/or storage of the medical solution. 3,4-dideoxy-glucosone-3-ene can be identified using the method “Identification and determination of 3,4-dideoxy-glucosone-3-ene ” and the toxicity of 3,4-dideoxy-glucosone-3-ene may be determined according to “In vitro assay for cytotoxicity”, both methods are described above under Materials and Methods.

Furthermore, the carbohydrate containing medical solution according to one embodiment of the present invention may comprise reduced levels of, or is substantially free from 3,4-dideoxy-glucosone-3-ene, and at least one detoxifying agent as defined above. This solution may also be completely free from 3,4-DGE if the detoxifying has been added to the carbohydrate containing medical solution before the degradation of the carbohydrates has been initiated or has proceeded to the production of 3,4-DGE. In a preferred embodiment the medical solution is a sterile carbohydrate containing medical solution, and the detoxifying agent may have been added prior to and/or after sterilisation.

The sterile carbohydrate containing medical solution according to one preferred embodiment of the invention may be comprised in a compartment container, particularly one adapted for peritoneal dialysis (PD), wherein the carbohydrate containing medical solution is present in one or more compartments and is substantially free of 3,4-dideoxy-glucosone-3-ene. One or more of the compartments comprise a carbohydrate, such as glucose and/or fructose, which during sterilisation and/or storage may be decomposed to form 3,4-dideoxy-glucosone-3-ene, and at least one detoxifying agent with the ability to prevent the generation of, reduce the level of or substantially eliminate by degrading, scavenging and/or modifying 3,4-dideoxy-glucosone-3-ene into a non-toxic compound. The sterile carbohydrate containing medical solution of the invention is preferably a heat sterilised peritoneal dialysis solution.

Method and Device for Identification and Determination of 3,4-dideoxy-glucosone-3-ene

In one of the embodiments of the invention the concentration of the 3,4-dideoxy-glucosone-3-ene is measured as an indication of the level of toxicity of the carbohydrate containing medical solution. The 3,4-dideoxy-glucosone-3-ene functions as a direct marker for the toxicity of the carbohydrate containing medical solution. Thus, detection of the marker implies that the carbohydrate containing medical solution is toxic and should thus not be used for treatment of mammals.

Other methods for identification of 3,4-dideoxy-glucosone-3-ene may be methods based on enzymatic reactions and/or colour reactions.

As to the device, the membrane or column according to other embodiments of the present invention have the ability to bind 3,4-dideoxy-glucosone-3-ene present in a carbohydrate containing medical solution.

The membrane may be a polymer membrane, such as a polymer membrane containing or coupled to one or more detoxifying agents on its surface, e.g. an amino, sulphite and/or sulphydryl compound. Alternatively, the polymer membrane may be made of a polystyrene divinyl benzene polymer, octadecyl silica or a combination thereof.

The column may be a polymer column made of a polystyrene divinyl benzene polymer, octadecyl silica or a combination thereof.

The membrane may be used for the embodiment according to the present invention in which a mammal having an undesired amount of 3,4-dideoxy-glucosone-3-ene in its body fluids, such as in the blood or in the abdominal fluids, is treated preferably extracorporeally. An example is a mammal suffering from diabetes. A 3,4-dideoxy-glucosone-3-ene binding agent, such as an amine, for example sulphydryl amino acid, may be coupled as a detoxifying agent to the membrane, whereupon the 3,4-dideoxy-glucosone-3-ene containing body fluid is brought into contact with the membrane, to substantially clear the body fluid from 3,4-dideoxy-glucosone-3-ene.

Apart from a membrane and a column other solid phase substrates may be used according to the present invention, examples of such substrates being beads, ELISA plates, test strips, diagnostic arrays or solid substrates for wound dressing or tissue engineering. The substrate may be any substrate of organic or inorganic origin for binding 3,4-dideoxy-glucosone-3-ene via the detoxifying agent, i.e. the 3,4-DGE binding agent. Examples of useful inorganic substrates are silica, modified glass or carbon (activated or non-activated).

In another embodiment the treatment may be an in vivo treatment, wherein a composition including a 3,4-dideoxy-glucosone-3-ene binding or detoxifying agent is injected into the patient to be treated for binding or detoxifying 3,4-dideoxy-glucosone-3-ene.

Method of Treatment of a Mammal Suffering from a Disease Condition Activating the Immune System

In one of its embodiments the present invention refers to a method of treating a mammal in need thereof, i.e. suffering from a disease condition activating the immune system in an adverse way with a solution containing 3,4-dideoxy-glucosone-3-ene in a non-toxic amount. In such a way, the immunosuppressive effect.of 3,4-dideoxy-glucosone-3-ene reduces the immune response, thereby ameliorating or eliminating the symptoms of the mammal.

Identification of 3,4-dideoxy-glucosone-3-ene and Determination of the Toxicity of 3,4-dideoxy-glucosone-3-ene

3,4-dideoxy-glucosone-3-ene was identified using a commercial standard 3,4-dideoxy-glucosone-3-ene preparation (Toronto Research Chemicals, Toronto, Canada). The HPLC system was a Hewlett Packard model 1100 equipped with a diod array detector (DAD). Two different separation columns were used. Method 1 used a Supelcosil C₁₈ column (Supelco, Bellefonte, Pa., USA) eluted with a gradient consisting of methanol-water. The flow was 1 ml/min, the gradient started with 5 min with 5% methanol and 95% water, from 5 min to 40 min the methanol content was linearly increased to 100% and then returned to 5% during 1 min, whereafter it was hold for another 5 min prior to next injection.

Method 2 used two Bio-Rad Aminex 87-H columns (Bio-Rad laboratories, Hercules, Calif., USA) coupled in series and eluted isocratically with 0.005M H₂SO₄ with a flow of 0.5 ml/min. The injection volume was for both methods 20 μL and the DAD detector was set to 230 nm. A standard curve was prepared in sterile filtered PD fluid and the content of 3,4-dideoxy-glucosone-3-ene in a PD solution was determined by measuring the peak height for 3,4-dideoxy-glucosone-3-ene in an autoclaved PD fluid and calculating the concentration. Method 1 was also used with a mass detector to confirm the identification by determination of the molecular mass.

Mouse fibroblast cells (L-929) were grown and incubated according to Järkelid et al., 2000, ATLA 28, 415-425. Inhibition of cell growth was determined for a 4% PD fluid with the following composition

| | | |
|---|---|---|
| glucose | 4.0% | |
| calcium | 1.75 | mM |
| lactate | 40 | mM |
| sodium | 132 | mM |
| magnesium | 0.5 | mM |

Additionally dose response curves were made in MEM (Minimal Essential Medium).

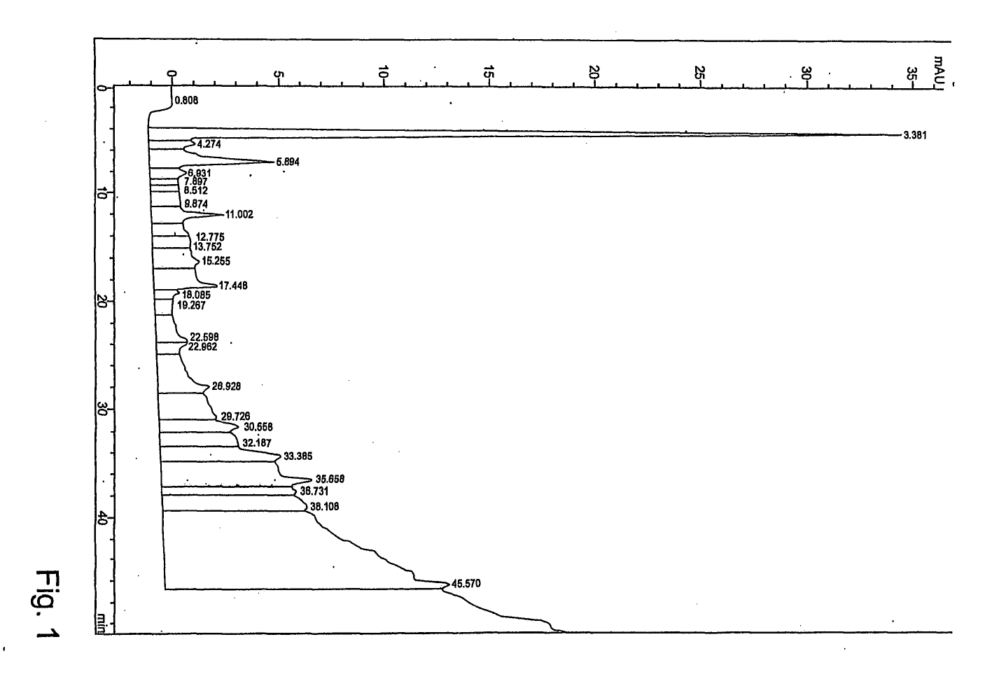

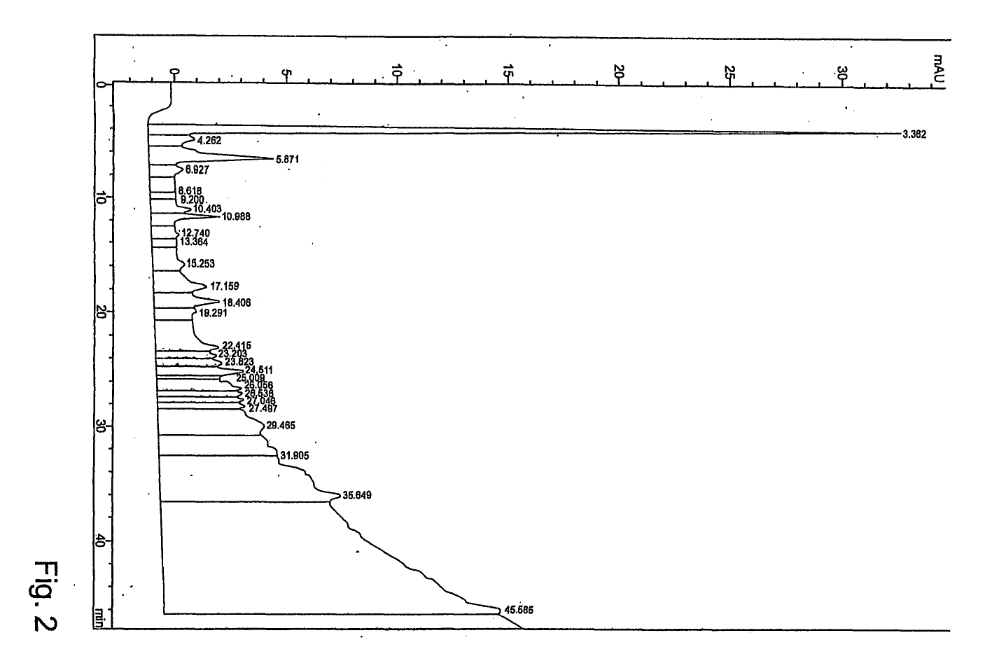

In the figures and the table below 3,4-dideoxy-glucosone-3-ene is abbreviated to 3,4-DGE. A chromatogram (FIG. 1) obtained with method 1 of a standard solution of 3,4-dideoxy-glucosone-3-ene shows one main and two minor peaks with absorption maximum at 228 or 233 nm. The retention time for the main peak was 5,894 min and for the minor peaks 4,274 and 35,658 min, the last peak being the trans form of 3,4-dideoxy-glucosone-3-ene. The chromatogram for a 4% PD fluid is presented in FIG. 2.

Using method 1 and a standard curve obtained with 3,4-dideoxy-glucosone-3-ene in a sterile filtered peritoneal dialysis (SF PD) fluid gives a concentration calculated on the main cis isomer of 23 μm 3,4-dideoxy-glucosone-3-ene.

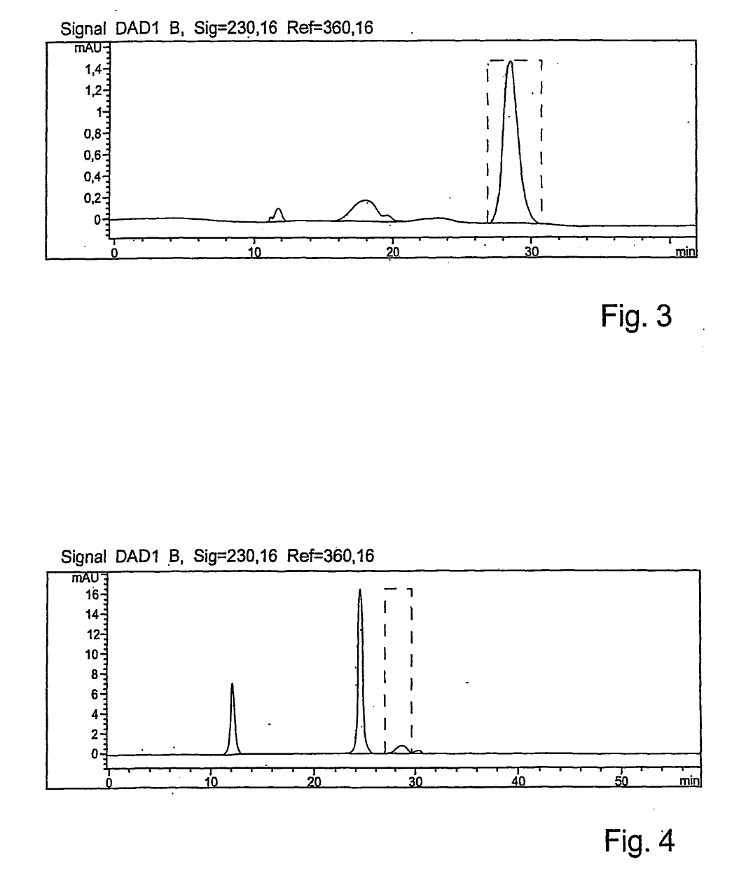

Method 2 was applied to obtain a different separation mechanism. FIG. 3 shows a chromatogram for a standard 3,4-dideoxy-glucosone-3-ene solution and FIG. 4 a corresponding chromatogram for a 4% PD solution. The concentration in 4% PD fluid obtained with this method was 19 μM.

When comparing peak heights for the different isomers in chromatograms it was clear that the proportions of the isomers were approximately the same in PD fluid and standard.

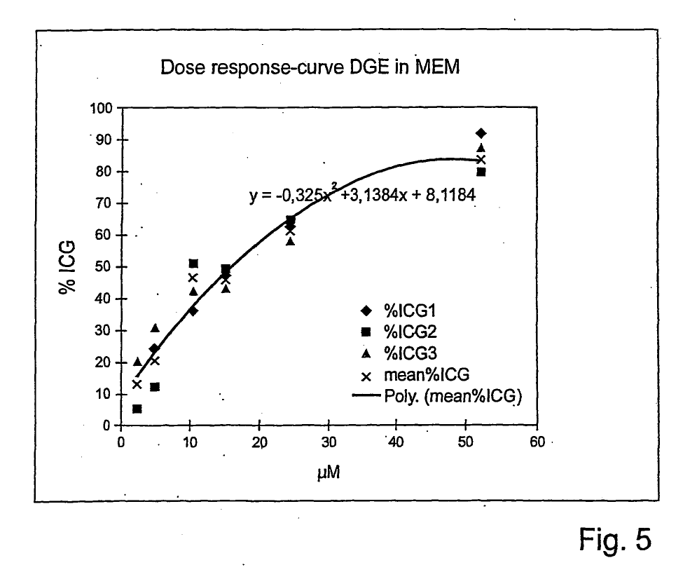

The results from incubation of L-929 cells with synthetic 3,4-dideoxy-glucosone-3-ene added in MEM are presented in table 1 below showing the values plotted in FIG. 5. The 4% PD solution was autoclaved while the PD fluid was sterile filtered. The PD solutions were tested twice (% ICG1 and % ICG2) and the mean ICG was calculated. Dose response curves in MEM (FIG. 5) gave an EC50 value of 16 μM.

A 4% PD solution holding 23 μM gave an ICG value of 72% which leads to the conclusion that 3,4-dideoxy-glucosone-3-ene is the major toxic agent in PD solutions.

TABLE 1
Dose response curve of 3,4-DGE in MEM

| Concentration | | | | | |
|---|---|---|---|---|---|
| of 3,4-DGE (μM), | MEM | % ICG1 | % ICG2 | % ICG3 | mean % ICG 2, 5 |
| | | | 5 | 20 | 12,5 |
| | 5 | 24 | 12 | 31 | 21,5 |
| | 10, 4 | 36 | 51 | 42 | 46,5 |
| | 15 | 47 | 49 | 44 | 46,5 |
| | 24, 3 | 63 | 65 | 58 | 61,5 |
| | 52 | 92 | 80 | 88 | 84 |
| 4 % PD solution | | 70 | 75 | | 72,5 |
| PD solution | | 30 | 22 | | 26 |

The results show in accordance with the commercial supplier's findings using thin layer chromatography that 3,4-dideoxy-glucosone-3-ene exists in different isomeric forms. In addition to the main cis isomer a possible minor cis isomer and also the trans isomer was found. These possible isomers can be identified both in standard solutions and in PD solutions.

The identification of 3,4-dideoxy-glucosone-3-ene in PD solutions using two independent chromatographic methods with diode array detection (DAD) and confirmation with LC-mass strongly supports its presence as a GDP.

The determination of 3,4-dideoxy-glucosone-3-ene was made using DAD detection. The spectrum was not showing a totally pure peak, but the influence of the impurity was estimated to be minor i. e. less than 10%. In addition the impurity was detected both in standard and in a PD solution, which may further decrease its influence on the accuracy of the method. Determination obtained with an ordinary UV detector showed comparable results as with DAD detection supporting its use for routine determination of 3,4-dideoxy-glucosone-3-ene.

3,4-dideoxy-glucosone-3-ene is the first reported glucose degradation product causing cytotoxicity in those concentrations found in PD solutions and by means of the present invention it is possible to discard carbohydrate containing medical solutions comprising said toxic compound prior to use on a mammal and reduce complications caused by 3,4-dideoxy-glucosone-3-ene.

The present invention is especially applicable in connection with and in the treatment of diseases with disturbed glucose metabolism or compromised removal of glucose degradation products, e.g. diabetes and uremia. The present invention also refers to use of 3,4-DGE for the production of a medical solution for the treatment of said disease conditions.

It may also be useful in combination with fluids used for blood donation purposes or blood component storage, since such fluids often contain glucose as a stabilising or nutritional agent for blood components. Moreover, it may be used in connection with carbohydrate, e.g. glucose, containing fluids for cell culture technology, e.g. when long term cultures for tissue engineering are considered.

Still further the present invention may be useful in connection with functional foods, preferably in the form of a fluid, in order to reduce or substantially eliminate the amount of 3,4-dideoxy-glucosone-3-ene in such functional foods, specifically in foods for diabetic patients or critically ill patients.

Method for the Production of 3,4-dideoxy-glucosone-3-ene

The present invention also refers to a new method for the production of 3,4-DGE at a low cost in laboratory scale, which will be described below, in contrast to known methods for the production of this compound resulting in a very expensive compound. In the initial extraction step of the inventive method 20 g of glucose dissolved in 500 ml of water was heated in an autoclave at 121° C. for 20 min. 500 ml of autoclaved glucose was pumped through a 5 g ENN solid phase extraction column (Sorbent) at a flow rate of 20 ml/min.

In the following purification step the column was then washed with 50 ml of Millipore water and eluted with 50 ml of 50% ethanol. The ethanol fraction was then freeze-dried to dryness and dissolved in water to give a solution holding 0.5 g/L (3.5 mM) of 3,4-dideoxy-glucosone-3-ene.

In such a way an ENV column can be washed with water with a view to avoiding disturbances and be cleaned from e.g. glucose, wherein 3,4-DGE is produced in a hitherto unknown way.

1. Method for detoxifying a carbohydrate containing solution, wherein a detoxifying agent is contacted with said solution in order to prevent the generation of, reduce the level of or substantially eliminate the amount of 3,4-dideoxy-glucosone-3-ene.

2. Method according to claim 1, wherein the carbohydrate containing solution is a medical solution, a cell cultivation fluid or food, preferably in fluid form.

3. Method according to any one of claims 1 and 2, wherein the carbohydrate containing solution is contacted either directly in solution with the detoxifying agent, optionally bound to beads or particles, for binding of or reaction with 3,4-dideoxy-glucosone-3-ene, or with a membrane or column having coupled thereto the detoxifying agent for binding of or reaction with 3,4-dideoxy-glucosone-3-ene to the column or membrane, and is withdrawn with a reduced amount of 3,4-dideoxy-glucosone-3-ene.

4. Method according to any one of claims 1-3, wherein the detoxifying agent is selected from the group consisting of substances containing amino, sulphite and/or sulphhydryl groups, preferably NaHSO₃, Na₂S₂O₅ or Na₂SO₃; a peptide structure or an amino acid, preferably polylysine or arginine; specific enzymes for the conversion of 3,4-dideoxy-glucosone-3-ene, and pharmaceutically acceptable substances giving a very low pH, preferably a pH of around 3, and, in the case when the detoxifying agent is bound to beads or particles or coupled to a membrane or column, it may also be selected from the group comprising polystyrene divinyl benzene polymers and octadecyl silica or a combination thereof.

5. Method according to claim 4, wherein the detoxifying agent is a monosulphite compound or a hydrogen sulphite compound in a concentration of 0.001-10 mM, preferably 0.001-0.2 mM, more preferably 0.001-0.1 mM, or a disulphite compound in a concentration of 0.0005-5 mM, preferably 0.0005-0.1 mM, more preferably 0.0005-0.05 mM.

6. Method according to any one of claims 1-5, wherein the carbohydrate is glucose, fructose, mannose, a polymer or derivative thereof, or mixtures thereof, preferably glucose.

7. Method according to any one of claims 1-6, wherein the carbohydrate containing solution is a sterile medical solution or a medical solution to be sterilised, preferably a dialysis solution, most preferably a peritoneal dialysis solution; and fluids for blood donation purposes or blood component storage.

8. Device for use in a method according to any one of claims 1-7 comprising a membrane or a column, having coupled thereto a detoxifying agent for the binding of 3,4-dideoxy-glucosone-3-ene, said membrane or column optionally being arranged between compartments in a multi compartment container or in tubes.

9. Device according to claim 8, wherein the detoxifying agent is a polystyrene divinylbenzene polymer or octadecyl silica or a combination thereof.

10. Method for detoxifying the body fluids of a mammal having 3,4-dideoxy-glucosone-3-ene in its body fluids, preferably the blood or the abdominal fluid, wherein the body fluids including 3,4-dideoxy-glucosone-3-ene is extracorporeally contacted with a membrane or column having coupled thereto a detoxifying agent as defined in claims 3 and 4 for binding 3,4-dideoxy-glucosone-3-ene.

11. Method for the treatment of a mammal suffering from disease condition adversely activating the immune system, wherein a non-toxic amount of 3,4-dideoxy-glucosone-3-ene is administered to the mammal as an immunosuppressive agent.

12. Method for identification and determination of the 3,4-dideoxy-glucosone-3-ene in a carbohydrate containing solution comprising determining both the presence and amount of 3,4-dideoxy-glucosone-3-ene in the solution, wherein said solution is subjected to HPLC.

13. Method for the production of 3,4-dideoxy-glucosone-3-ene, wherein a carbohydrate, preferably glucose, is dissolved in water, the solution so obtained is heated and thereafter extracted through a column in which 3,4-dideoxy-glucosone-3-ene produced in the glucose containing solution is bound to the column, followed by eluation and obtaining the 3,4-dideoxy-glucosbne-3-ene.

14. A carbohydrate containing solution, preferably a medical solution, comprising a detoxifying agent as defined in claims 3 and 4.

15. Carbohydrate containing solution according to claim 14, wherein the carbohydrate is glucose, fructose, mannose, a polymer or derivative thereof, or mixtures thereof, preferably glucose.

16. Carbohydrate containing solution according to any one of claims 14 and 15, wherein the carbohydrate containing solution is a heat sterilised medical solution.

17. Carbohydrate containing solution according to any one of claims 14-16, wherein the carbohydrate containing solution is a peritoneal dialysis solution.

18. A compartment container comprising a carbohydrate containing solution according to any one of claims 14-17.

19. A membrane or column comprising a 3,4-dideoxy-glucosone-3-ene binding agent as defined in claims 3 and 4 for binding 3,4-dideoxy-glucosone-3-ene.

20. Use of a 3,4-dideoxy-glucosone-3-ene detoxifying agent for the production of a medical solution, preferably a dialysis solution, containing carbohydrates for the treatment of diseases involving a disturbed carbohydrate mechanism of compromised removal of carbohydrate degradation products.

21. Use according to claim 20, wherein the carbohydrate containing solution is contacted directly in solution with the detoxifying agent, optionally bound to beads or particles, for binding of or reaction with 3,4-dideoxy-glucosone-3-ene, or with a membrane or column having coupled thereto the detoxifying agent for binding of or reaction with 3,4-di-deoxy-glucosone-3-ene to the column or membrane, and withdrawing the carbohydrate containing medical solution with a reduced amount of 3,4-dideoxy-glucosone-3-ene.
